(19) United States
(12) Patent Application Publication
(10) Pub. No.: US 2010/0256668 A1
(43) Pub. Date: Oct. 7, 2010

(54) DEVICES FOR TREATING THE EXTERIOR OF ANATOMICAL STRUCTURE, AND METHODS USING THE SAME

(75) Inventors: Kenneth Eugene Perry, JR., Bainbridge Island, WA (US); Paul E. Labossiere, Bainbridge Island, WA (US)

Correspondence Address: GRAYBEAL JACKSON LLP, 400 - 108TH AVENUE NE, SUITE 700, BELLEVUE, WA, 98004, US

(73) Assignee: Echobio LLC, Bainbridge Island, WA (US)

(21) Appl. No.: 12/442,069

(22) PCT Filed: Sep. 19, 2007
(86) PCT No.: PCT/US07/20363

§ 371 Date: Jun. 16, 2010

Related U.S. Application Data
(60) Provisional application No. 60/845,940, filed on Sep. 19, 2006.

(51) Int. Cl. A61M 29/00

(52) U.S. Cl. 606/194

(57) ABSTRACT

Systems, devices, methods, etc., comprising radial pressure devices applied to the exterior of anatomical structures such as blood vessels, typically for inhibition and/or treatment of aortic aneurysms, as well as methods of making and deploying such systems and devices, etc. The devices, etc., generally comprise one or more exovascular cuffs for generating desirable mechanical forces, and may additionally comprise body structures for covering, containing or treating tissues. Also provided are other systems and devices for fixing the devices discussed herein or other implantable devices, typically used in conjunction with such devices, to the vessels. The devices, etc., may be deployed by open or minimally invasive techniques, including translumenal, exovascular and endovascular deployment methods.

### BACKGROUND

Aortic aneurysm repair has traditionally been managed with open resection and interposition of a Dacron tube graft. Many subjects who present with this disorder have other serious systemic disease states such as coronary artery disease, chronic renal failure, diabetes mellitus, cerebrovascular disease, and obstructive pulmonary disease. These co-existing diseases may lead to postoperative complications to include ventilator dependence, renal failure, myocardial infarction, stroke, and death. More recently, minimally invasive endovascular approaches to aortic aneurysm repair (EVAR) have been pursued due to their potential for significantly reducing procedure related mortality and morbidity, as well as expected faster recovery times and reduced costs through decreased use of hospital resources.

While the long term benefits of minimally invasive EVAR treatments have yet to be defined, these approaches appear to offer a short-term benefit over open repair for the management of large abdominal aortic aneurysms (AAA) [3,4]. However, data from registries, e.g., EUROSTAR (European Collaborators Registry on Stent-graft Techniques for AAA Repair) and RETA (Registry for Endovascular Treatment of Aneurysms) [5,6], indicate the desire for close surveillance of endografts over many years. Complications arise in 25-40% of patients who often need additional interventions or conversion to open surgery [7].

Aneurysmal neck dilation, migration, and endoleak are all inter-related problems that been reported thus far to limit the success of EVAR. Similar problems complicate stents for thoracic aneurysm.

One previous attempt to develop an approach for the treatment of aortic aneurisms involving an exovascular approach achieved only minimal success [8]. In that study, an open surgical procedure was used.

The present devices, systems, methods, etc., reduce one or more shortcomings or limitations of such EVAR approaches as well as other concerns for the treatment of aortic aneurysm repair, for example by providing external support solutions that address at lest one of these problems.

### BRIEF SUMMARY

The devices, systems, methods, etc., herein comprise at least one force-applying body member that is deployed according to the methods herein to the exterior of anatomical structures in order to provide a pre-determined magnitude, orientation and spatial distribution of compressive radial (inwardly directed) forces to said anatomical structures. The anatomical structures to which these devices are applied are typically hollow body organs, for example lumens such as blood vessels and parts of the gastrointestinal tract. More typically the anatomical structure is the aorta, wherein the devices, systems, etc., herein are used for the prevention or therapeutic treatment of aortic aneurysms. The devices, etc., are configured to be deployed typically using minimally invasive surgical procedures including laparoscopic access methods, endoscopic access methods (e.g., involving translumenal deployment), and natural orifice access methods, although the devices may also be readily used in open surgical procedures. The devices herein may be used alone or in combination with other therapeutic devices implanted in the body, such as endolumenal stents, embolic coils, artificial heart valves, gastrointestinal implants, and the like. In the case of treating aortic aneurysms, the methods and devices, etc., herein can be advantageous, for example, for inhibiting, minimizing and containing the expansion of existing aneurysms, endoleaks following stent deployment, stent migration, and can also be advantageously used for extralumenal drug delivery. The devices, systems, etc., can also serve for fixation or support for other therapeutic devices such as sensors or transducers used in the treatment of aneurysms.

The devices herein are configured to wrap around, encircle or otherwise at least substantially surround the anatomical structures to which they are applied, thereby providing a specifically controlled compressive radial force to a desired portion of the outside wall or external surface of said anatomical structures. “Specifically controlled” indicates the compressive radial force is specifically selected and implemented by the device, it is not random nor inherent forces that are applied via the attachment of any device to a structure. In one embodiment, the devices herein are provided in an initial collapsed configuration and are configured to be re-configured in a self-actuating manner to a second expanded or deployed configuration. This can allow the devices to be introduced into the body using simple surgical applicators having minimal size, while allowing larger anatomical structures to be treated.

In some embodiments, the compressive radial force is configured to be applied substantially uniformly around the exterior of the anatomical structure, whereas in other embodiments the devices may be configured to provide a specifically controlled variation in compressive radial force depending upon the radial position around the anatomical structure. The compressive radial forces produced by the devices may be fixed during the design and manufacture of the device, in which case, for example, devices having different sizes, shapes and force characteristics may be supplied as part of a surgical kit herein. In other embodiments, additional features and mechanical elements may be incorporated in the devices that allow the compressive radial force to be varied and/or adjusted during or after placement of the device in order to achieve optimal therapeutic results. Examples of such features and elements include springs, hinges, cams, position dependent shape and dimensions, use of different materials or tailored materials, and so on.

Generally, the at least one force applying body member comprises a substantially flexible wire, rib, strap, band, sheet, wrap, cuff or similar curved mechanical element that is deployed circumferentially around the target anatomical structure to deliver the desired compressive radial forces to the underlying tissue. Non-flexible versions with ratchets or other closure mechanisms can also be used. These mechanical elements can be manufactured from any suitable flexible biocompatible material having suitable characteristics, including but not limited to stainless steel, titanium, polymers, superelastic NiTi alloy, and combinations of the foregoing.

In certain embodiments, described in detail below, two or more such force applying body members are deployed around the target anatomical structure in order to additionally provide a desired compressive radial force profile along a larger axial length of the anatomical structure, where the radial force profile may be either constant or variable along said axial length. Combined with compressive radial forces that may be configured to vary circumferentially (as described above), the ability to adjust the compressive radial force profile along axial length allows devices to be tailored to deliver true three dimensional radial force profiles to the anatomical structure, thereby achieving improved therapeutic results, especially in cases where the target anatomical structure exhibits variability in shape, size or tissue characteristics over the region where treatment is desired. It is also possible to use multiple force applying body members having different sizes such as different widths, different diameters, etc., and compressive radial force characteristics in order to accommodate variability in the size or other characteristics of the underlying tissue as a function of position.

The force applying body members herein may optionally include other features and/or mechanical elements that can be gripped, grasped, held, moved, and so on, either by hand, by using conventional surgical tools (graspers, forceps, retractors, and the like) or by using custom surgical tools that may be optionally provided as part of the systems herein. These features and/or elements are configured to assist with the initial deployment, positioning, repositioning after initial placement, and removal of the devices. Examples of such features and elements include arms, loops, hooks, notches, grooves, holes, and the like. Mechanical latches, closures, interconnects, springs, fasteners and the like may optionally be included to limit the outward expansion of the device or apply a more specific localized force to the underlying tissue.

In embodiments involving two or more force applying body members, the individual force applying body members may be at least partly connected to one another by one or more optional connecting members. The optional connecting member not only serves to operatively interconnect the two or more force applying elements (which can simplify delivery, deployment and adjustment of the device, and so on) but the optional connecting member may provide additional compressive radial forces to the underlying tissue, and may include other features to aid in positioning, securing and/or adjusting of the devices. The optional connecting member may be rigid, flexible and combinations thereof, and may be provided initially as part of the device assembly or it may be delivered independently and subsequently fixedly attached to the two or more force applying members during or after their deployment. In one embodiment, the optional connecting member is produced from a flexible biocompatible material such as cloth, mesh, fabric, sheet, tube, and the like. In another embodiment, the optional connecting member is produced by injecting material into spaces between and/or surrounding the two or more force applying members. Various known approaches may be used to attach the optional connecting member to the two or more force applying members, including but not limited to sutures, stitches, clips, staples, rivets, friction fits, mechanical connectors, and combinations thereof.

Both the force applying members and optional connecting members herein may further incorporate additional features and/or mechanical elements located on at least a portion of the tissue contacting surfaces that are configured to grip, grasp, mate, hold, attach or otherwise anchor the devices herein to the underlying tissue. Examples of such features and elements include projections, hooks, barbs, serrations, frictional coatings, and the like. These features and mechanical elements can prevent slippage or other unintended movements of the devices after placement, ensuring proper positioning and orientation are maintained.

Both the force applying members and optional connecting members herein may further incorporate at least one additional therapeutic element for providing therapy to the treatment site. For example, coatings may be provided on the devices to controllably release drugs, electrical components may be included to stimulate the underlying tissue, and sensors may be incorporated to detect, record and/or transmit important scientific information about the treatment site to clinicians.

At least a portion of the devices herein may optionally be shaped, contoured or otherwise configured to interact with other therapeutic devices implanted in the body. For example, the devices may be configured to interact with endoluminal stents placed inside blood vessels for the treatment of aortic aneurysms or other vascular irregularities. By applying a controlled compressive radial force to the exterior of the vessel, the devices herein effectively increase fixation, prevent migration, minimize leakage and otherwise improve the performance of the implanted endoluminal device.

The discussion herein provide definitions of some of the terms used herein. All terms used herein, including those specifically discussed below in this section, are used in accordance with their ordinary meanings unless the context or definition clearly indicates otherwise. Also unless expressly indicated otherwise, the use of “or” includes “and” and vice-versa. Non-limiting terms are not to be construed as limiting unless expressly stated, or the context clearly indicates, otherwise (for example, “including,” “having,” and “comprising” typically indicate “including without limitation”). Singular forms, including in the claims, such as “a,” “an,” and “the” include the plural reference unless expressly stated, or the context clearly indicates, otherwise.

The scope of the present devices, systems and methods, etc., includes both means plus function and step plus function concepts. However, the claims are not to be interpreted as indicating a “means plus function” relationship unless the word “means” is specifically recited in a claim, and are to be interpreted as indicating a “means plus function” relationship where the word “means” is specifically recited in a claim. Similarly, the claims are not to be interpreted as indicating a “step plus function” relationship unless the word “step” is specifically recited in a claim, and are to be interpreted as indicating a “step plus function” relationship where the word “means” is specifically recited in a claim.

These and other aspects, features and embodiments are set forth within this application, including the following discussion and drawings. In addition, various references are set forth herein, including those below that discuss certain systems, apparatus, methods and other information; all such references are incorporated herein by reference in their entirety and for all their teachings and disclosures, regardless of where the references may appear in this application.

### BRIEF DESCRIPTION OF THE FIGURES

FIG. 1 shows a device according to one embodiment herein: (A) cross-sectional overview, (B) perspective view of a deployed configuration.

FIG. 2 shows a device according to another embodiment herein comprising a coil spring: (A) cross-sectional overview, (B) perspective view of a deployed configuration.

FIG. 3 shows a top plan view of a device according to another embodiment herein comprising an asymmetric coil spring.

FIG. 4 shows a perspective view of a device according to another embodiment herein comprising two devices deployed on a blood vessel in the deployed configuration.

FIG. 5 shows a perspective and cross-sectional view of a device according to another embodiment herein wherein the device has a specifically shaped cross-section.

FIG. 6 shows a cross-sectional view of a device according to another embodiment herein wherein the device further comprises reverse-curved ends to decrease point pressure on the anatomical structure at the end points of the device and a retainer structure.

FIG. 7 shows a system herein comprising a medical pressure device and a minimally invasive applicator device: (A) perspective overview, (B) cross-sectional view of a close up of distal end.

### DETAILED DESCRIPTION OF THE INVENTION

Turning first to a general discussion of certain aspects of the methods, devices, systems, etc., discussed herein, in one aspect, such systems, devices, methods, etc., provide medical pressure devices configured to apply a specifically controlled, compressive radial force to the exterior of an anatomical structure, the medical pressure device comprising at least one collapsible force-applying body member comprised of a surgically acceptable material, the force-applying body member having at least a first collapsed shape and a second expanded shape. The first collapsed shape can be sized for deployment by a minimally invasive applicator device, and the second expanded shape can comprise a substantially U-shape configured to at least substantially encircle the anatomical structure and to provide a specifically controlled magnitude, orientation and spatial distribution of compressive radial force to the exterior of the anatomical structure. A U-shape generally means a shape open on one side and closed on other sides and includes devices that in cross-section may be oblong, circular, square, etc.

In one embodiment, the force-applying body member can comprise at least one collapsible element disposed between at least two substantially opposed arms. In this and other embodiments (Unless expressly stated otherwise or clear from the context, all embodiments, aspects, features, etc., can be mixed and matched, combined and permuted in any desired manner.) The force-applying body member can be resiliently flexible, can comprise at least one collapsible element disposed between at least two substantially opposed, substantially rigid arms or have other configurations. The medical pressure device can be curved to partially, completely or almost completely encircle the anatomical structure such as an aorta, abdominal aorta, thoracic aorta, lumen of the gastrointestinal tract or peripheral vasculature.

The device can comprise at least one cooperative element configured to cooperatively interact with at least one other therapeutic device implanted in the anatomical structure such as an endolumenal stent, an embolic coil, an artificial heart valve, and a gastrointestinal implant. The minimally invasive applicator device can be at least one of an endoscope and a catheter. Generally speaking, an “endoscope” can be a generally tubular device for insertion into a body, typically via canals, vessels, passageways or body cavities for any of a variety reasons, such as diagnostic purposes, the injection or withdrawal of fluids or to keep a passageway open. As used herein, an endoscope can be an in vivo optical viewer for viewing internal targets (such as internal organs) and includes other such internal, in vivo optical viewers such as laparoscopes, fundascopes, colposcopes, otoscopes and surgical microscopes. An endoscope can be similar to a catheter, except that generally an endoscope can be considered to transmit an image while a catheter does not; for the purposes of the present specification, the term endoscope includes catheter unless otherwise clear from the context. The endoscope or catheter can be typically rigid, but can be flexible, and can be lighted or unlighted. The discussion herein regarding endoscopes also generally applies to other types of in vivo optical viewers, including viewers for external use such as otoscope-like viewers for examining the skin, unless clear from the context.

The collapsible element can be self-actuating such that it converts from the first shape to the second shape when the restraining pressure can be removed without external input other than the removal of the restraining pressure. The collapsible element can be a coil spring, which can be symmetrical or asymmetrical. The force-applying body member can comprise at least two substantially opposed arms, wherein at least one of the arms can comprise at least one inwardly-facing anchoring element configured to engage the anatomical structure. Each arm can comprise an equal or unequal number of inwardly-facing gripping elements. The at least one inwardly-facing anchoring element can comprise at least one of a projection, hook, barb, serration, tooth, frictional coating and glue. The at least one inwardly-facing anchoring element can be configured to releasably or permanently engage the anatomical structure.

The force-applying body member can comprise at least two substantially opposed arms, wherein a first arm can comprise a retainer structure configured to interact with a second arm to retain the medical pressure device to the anatomical structure. The retainer structure can comprise reverse-curved tips on each of the arms, the reverse-curved tips extending away from the anatomical structure and sized to retain a suture extending between the reverse-curved tips, or a suture permanently attached a first arm and configured to be attached to a retaining structure of a second arm. The retainer structure can comprise at least one of a suture, tie, spring, latch, interlocking element, or ratchet. The compressive radial force can be configured to be applied substantially uniformly or non-uniformly throughout the medical pressure device to the anatomical structure.

The medical pressure device can be at least one of a substantially flexible wire, rib, strap, band, sheet, wrap or cuff, and can comprise at least one substantially opaque marker that can be substantially opaque to at least one scanning visualization modality. The force-applying body member can be composed of at least one of stainless steel, titanium, polymers, and superelastic NiTi alloy. The medical pressure device can also comprise at least one laterally-disposed interlocking element configured to interlockingly connect the medical pressure device to a second medical pressure device, and/or at least one connecting member configured to connect the medical pressure device to a second medical pressure device. The connecting member can comprise at least one flexible biocompatible material comprising at least one of cloth, fabric, mesh, sheet and screen. The medical pressure device can further comprise at least one additional therapeutic element configured to provide a treatment to the anatomical structure in addition to the radial pressure, such as a coating, an electrical stimulator and a sensor. The coating can comprise a controllably released drug, the electrical stimulator component can be configured to stimulate the underlying anatomical structure, and the sensor can be configured to detect, record and/or transmit information about the anatomical structure to clinicians.

In another aspect, the methods, etc., herein include using a medical device to apply a specifically controlled, compressive radial force to the exterior of an anatomical structure, the method comprising: providing at least one collapsible force-applying body member comprised of a surgically acceptable material in a first, collapsed shape, expanding collapsible force-applying body member to a second, expanded shape comprising a substantially U-shape configured to at least substantially encircle the anatomical structure, and applying the force-applying body member to the exterior of the anatomical structure to provide a specifically controlled magnitude, orientation and spatial distribution of compressive radial force to the exterior of the anatomical structure.

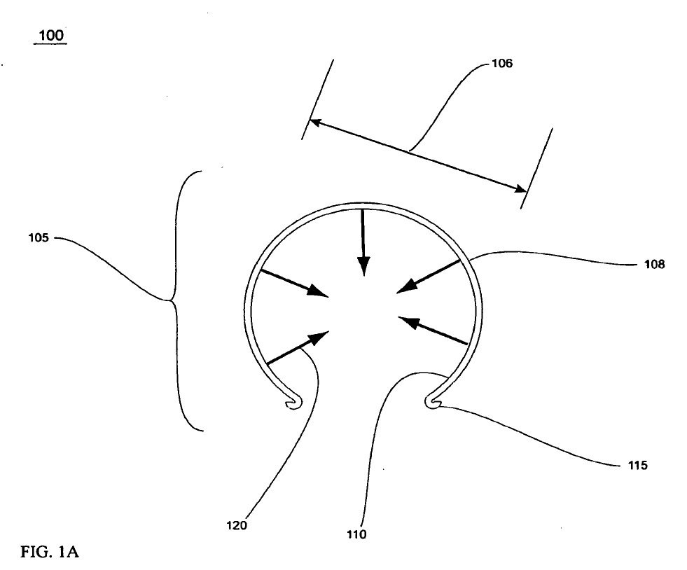

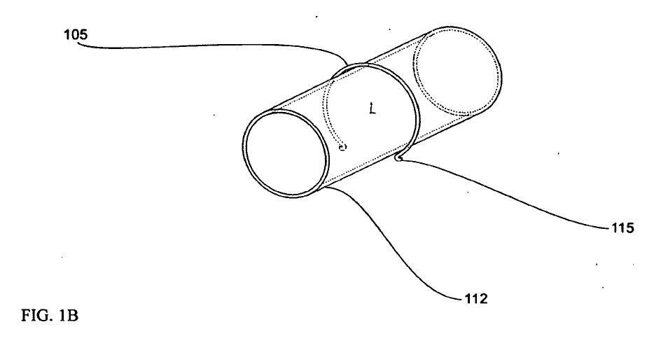

Turning to a discussion of the exemplary embodiments in the Figures, a radial pressure device according to one embodiment herein is illustrated in FIG. 1. In FIG. 1A, radial pressure device 100 comprises force applying member 105 having a generally circumferential and non-continuous shape, being sized appropriately for at least substantially enclosing the target anatomical structure. Force applying member 105 may be produced from shaped wire, strip, rod, tubing, or the like, and can be made of any flexible biocompatible material having suitable characteristics. Typically force applying member 105 is produced from a highly elastic engineering material such as spring steel, titanium or structural polymer, and most typically it is produced from a superelastic material such as NiTi alloy (e.g. Nitanol) or superelastic polymer. The diameter 106 of force applying member when applied to the thoracic aorta is generally between about 30-120 mm, typically between about 35-80 mm, and more typically between about 40-70 mm. The diameter 106 of force applying member when applied to the abdominal aorta is generally between about 25-70 mm, typically between about 30-70 mm, and more typically between about 30-65 mm. The diameter 106 of force applying member when applied to an iliac artery is generally between about 5-40 mm, typically between about 7-25 mm, and more typically between about 8-20 mm.

Force applying member 105 is configured with outward facing surface 108 and tissue contacting surface 110, the latter of which is located toward the inside of force applying member 105 and being configured for making substantially intimate contact with the anatomical structure 112 when the device is deployed according to the methods herein, as shown in FIG. 1B. Curved tips 115 positioned at each of the free ends of force applying member 105 allow device 100 to be grasped, held, moved and/or adjusted for the purposes or initially deploying, positioning, re-positioning or removal of device 100. Curved tips 115 are typically formed, bent, rounded or otherwise configured such that no points, tips, ends or other sharp features come into direct contact with the target tissue, thereby minimizing the possibility for tissue damage during or after deployment of the device.

When sized appropriately relative to the anatomical structure to which device 100 is to be applied, and due to the flexible elastic properties of force applying member 105, compressive forces 120 acting substantially in the radial direction are transmitted by tissue contacting surface 110 to the underlying tissue to which intimate contact is established during placement of device 100. Depending upon the specific geometry, dimensions, and material properties, each of which may be optimized within the scope herein, both the magnitudes and orientations of compressive forces 120 may be adjusted to achieve the desired therapeutic results. Compressive forces 120 need not be uniform along the length of tissue contacting surface 110, and the ability to further tailor the above mentioned variables to generate higher and/or lower forces at specific locations, or to alter the directions of said forces, being applied to the target anatomical structure are considered within the scope herein. The diameter 106 of force applying member may be sized to effect a reduction in the size of the target anatomical structure between about 3-30%, typically between about 5-20%, and possibly between about 7-15%.

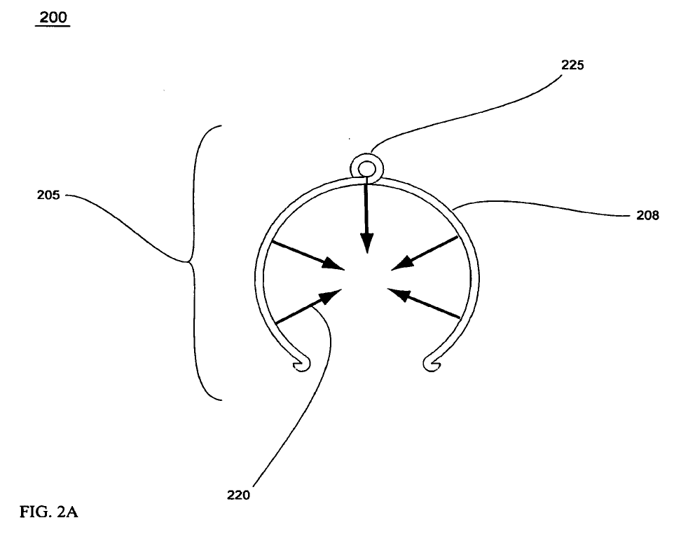

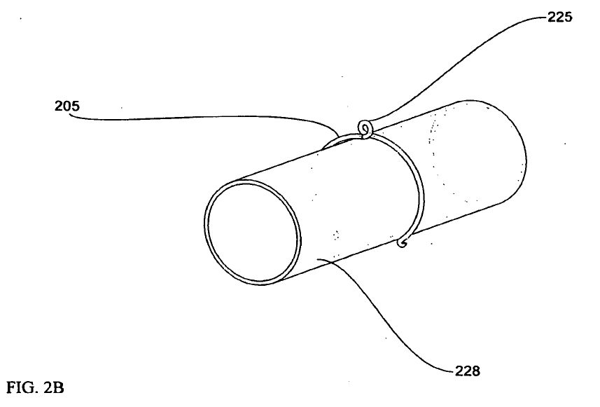

FIG. 2 shows another embodiment herein. Device 200 is similar to device 100, however in device 200 force applying member 205 further incorporates an optional force adjustment mechanism. Such a force adjustment mechanism can be used to aid in the deployment or positional adjustment of device 200 (e.g. to allow a greater range of inward motion or limit outward expansion) or to achieve higher compressive forces than would otherwise be possible based on the geometry, dimensions and material properties of force applying member 205. A variety of force adjustment mechanisms may be optionally incorporated in device 200, using design features and mechanical elements having suitable characteristics. Examples of such force adjustment mechanisms include linear springs, coil springs, gears, screws, levers, cams, ratchets, hinges, latches, and the like. In the example provided (FIG. 2) the force adjustment mechanism comprises coil spring 225 extending outward from outward facing surface 208 and positioned approximately midway along the length of force applying member 205. Coil spring 225 is configured to resist the outward expansion of force applying member 205, while increasing the magnitude of compressive forces 220 applied to the anatomical structure 228 (FIG. 2B). The diameter and number of turns used in coil spring 225 can be adjusted to optimize these characteristics.

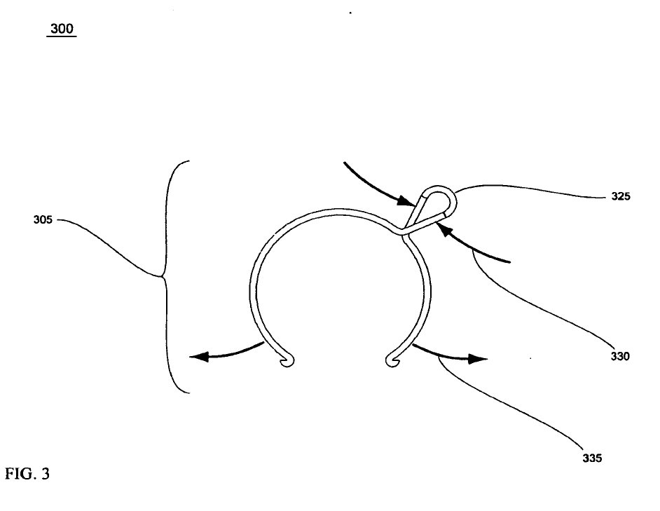

FIG. 3 shows another embodiment herein in which an optional force adjustment mechanism additionally incorporates elements for opening or releasing the compressive forces generated by the device, to further aid in placement, positioning, repositioning or removal of the device during use. In this example device 300 is similar to device 200, however spring element 325 has been configured asymmetrically. In this manner, by pressing the sides of spring element 325 using hand forces, forceps, graspers or other similar surgical tools, as indicated by 330, an expansion (i.e. opening) force 335 is generated that counteracts the self-actuating compressive forces and causes the diameter of force applying member 305 to increase. This partially or completely offsets the compressive forces applied to the anatomical structure allowing the position of the device to be adjusted. If the diameter of force applying member 305 is increased further in this manner, device 300 can be opened sufficiently to allow initial placement around (e.g., deployment) or removal of the device from the anatomical structure. Therefore, by incorporating additional elements of grasping and releasing the compressive forces as part of (or in conjunction with) an optional force adjustment elements, devices herein can conveniently be placed, adjusted and removed using minimally invasive techniques. A variety of features and mechanical elements having suitable characteristics may be used for such purposes and are considered within the broad scope herein.

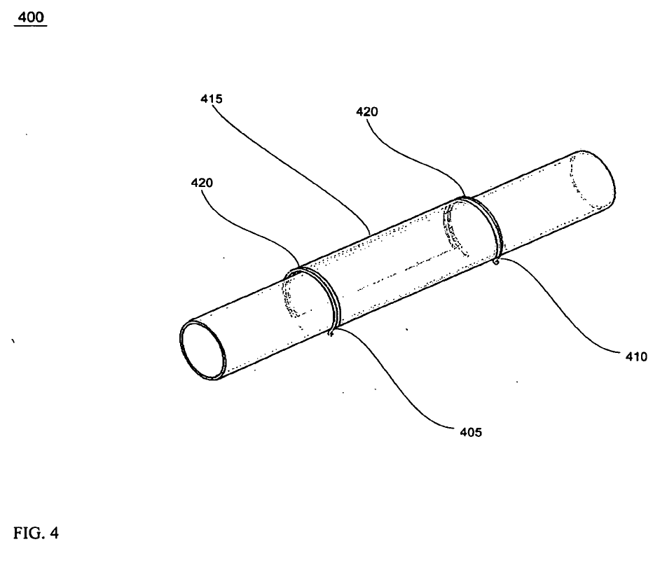

In other preferred embodiments, it may be desirable to place two or more force applying members to a specific treatment site to deliver improve therapeutic results over a larger area of the target anatomical structure, as illustrated in FIG. 4. Device 400 comprises two force applying members 405 and 410 (as previously described) that have been placed in close proximity to one another around an exemplary anatomical structure such as a blood vessel. The number, spacing, and force profile of the two or more force applying members can be fixed or variable, and in some situations can be adjusted by the surgeon during deployment to tailor the treatment to the specific site and particular anatomical variations of the individual patient.

Also shown in FIG. 4 is the incorporation of optional connecting member 415 which is positioned between and fixedly connected to force applying members 405 and 410. Optional connecting member 415 is a substantially circumferential element that spans at least the distance between force applying members. It may be rigid, flexible and combinations thereof. Optional connecting member 415 may be of fixed size and shape, serving primarily as a spacer and/or support structure for the force applying members.

Optional connecting member 415 may be a laterally-disposed interlocking element configured as a part of the force applying members and configured to connect the radial pressure device to a second radial pressure device, or may be an element added between two or more radial pressure device as described herein.

In other embodiments optional connecting member 415 may be configured to prevent outward expansion and therefore be used to provide a restraining function for the tissue located between the force applying members. In yet other embodiments, optional connecting member 415 may also be configured and configured to provide additional compressive radial force to the treatment site. In certain preferred embodiments, optional connecting member 415 is produced from a flexible biocompatible material such as cloth, fabric, mesh, sheet, screen, and the like, which may be produced from organic fibers, metals, polymers, and combinations of the foregoing.

Optional connecting member 415 may be initially attached to the two or more force applying members, such that the entire device 400 is deployed by the surgeon as a single unitary structure. In such cases, the connections between optional connecting member 415 and the force applying members 405 and 410 are produced during manufacturing of the device assembly. In one example, force applying members 405 and 410 are flexible wire elements molded directly into a plastic optional connecting member. In other embodiments, optional connecting member 415 may be provided as an impendent component that is deployed separately, either during the same procedure that force applying members 415 and 410 are deployed, or at a later time. In such cases, optional connecting member 415 may be attached to the previously deployed force applying members 405 and 410 during placement by the surgeon using various well established surgical fixation and attachment elements. In either case, a variety of approaches may be used to attach the optional connecting member to the two or more force applying members, including but not limited to sutures, stitches, clips, staples, rivets, friction fits, mechanical connectors, and combinations thereof. In the example shown, optional connecting member 415 comprises a synthetic fabric (i.e. a graft) and force applying members 405 and 410 have been sewn into tubular sleeves 420 positioned at either end of the graft.

In other preferred embodiments where optional connecting member 415 is deployed after force applying members 405 and 410 are deployed, optional connecting member 415 may be formed by injecting a material into the spaces between and surrounding force applying members 405 and 410. In certain such embodiments, the injected material may be a fluid (liquid, gas and combinations thereof) that fills a pre-defined space such as a pouch, bladder, tubular structure, or the like, wherein the properties of the fluid and the filling pressure may be controllably adjusted to achieve desired retention or force applying characteristics of optional connecting member 415. In yet other such embodiments, the injected material may be a liquid, epoxy or gel-like substance that can be cured, transformed, hardened or whose properties may otherwise be modified after injection to achieve desired retention or force applying characteristics of optional connecting member 415.

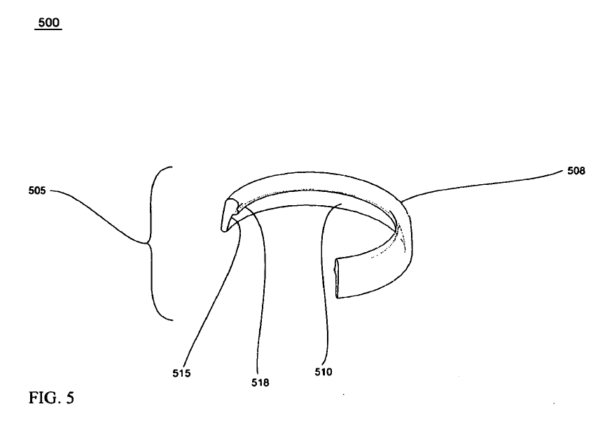

In other embodiments, individual force applying members may be configured to provide the desired compressive radial forces over a larger surface area of the target anatomical structure, as illustrated in FIG. 5. Device 500 comprises force applying member 505 having outward facing surface 508 that is produced from a molded polymer and provides a significantly larger surface area of tissue contacting surface 510, thereby essentially serving the combined functions described previously for the force applying member and optional connecting member. Also shown in this example, the cross section of force applying member 505 is optionally produced having a customized shape, and tissue contacting surface 510 exhibits a contoured surface profile 515. Contoured surface profile 515 may be configured to conform to a variable shape of the underlying tissue, it may be used to produce higher compressive stresses in a specific tissue location, or it may be configured to interact in a desired manner with another therapeutic device that may be implanted inside the anatomical structure. In the example shown, ridge 518 is provided as part of contoured surface profile 515. Relevant examples of such use involves contour surface profile 515 being provided in the form of ridges, protrusions or other male features that are intended to nest within or mate with grooves, indentations or other female features that may be provided on the exterior surface of an endoluminal device (not shown) such as a stent, stent graft, artificial heart valve, or the like. The conforming features provided on opposite sides of the wall of the anatomical structure can serve to prevent leaks, slippage and other known complications associated with the such endolumenal devices. Molded bodies having various shapes, sizes, contact surface, areas, contoured profiles are considered within the broad scope herein.

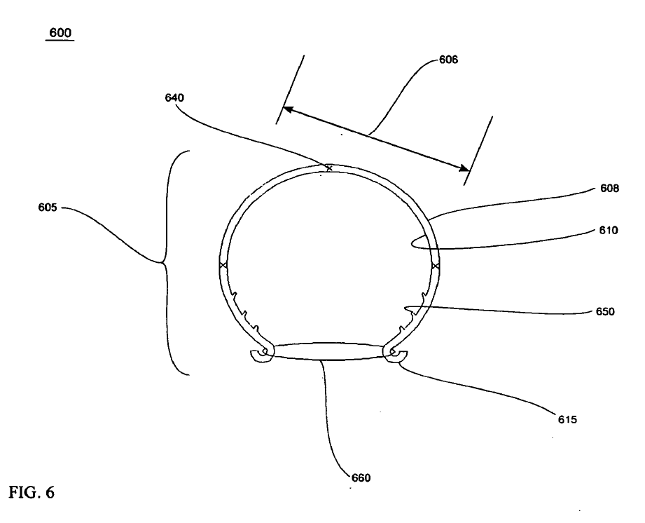

A wide variety of other functional features and mechanical elements may optionally be included in the devices herein in order to enhance therapeutic performance, increase safety or enhance the ease-of-use, and such optional variations are considered within the broad scope herein. Some examples of such features are illustrated in FIG. 6. Device 600 comprises force applying member 605 having diameter 606, outward facing surface 608, tissue contacting surface 610, and reverse-curved tips 615. Also incorporated as part of force applying member 605 are substantially opaque markers 640. Markers 640 are substantially opaque to at least one scanning visualization modality such as x-ray, magnetic resonance, etc. (in other words, the markers are substantially opaque to at least one scanning visualization modality and may or may not be opaque to human vision), and can aid in the observation of the detailed shape, position and other performance-related aspects of device 600 for example during initial placement and afterward, using detection, diagnostic and visualization elements having suitable characteristics; for example, x-ray, magnetic resonance and similar imaging methods may be used. Markers 640 may be attached to the external surface or embedded within force applying member 605.

Also incorporated in device 600 are anchoring barbs 650 disposed on and projecting inward from tissue contacting surface 610 of force applying member 605. Barbs 650 are configured to slide easily over the external surface of the anatomical structure in the direction of motion that occurs on deployment, and then penetrate, grasp, grab, attach or otherwise anchor device 600 to the underlying tissue to minimize or prevent undesirable motions after placement. Various other types of anchoring features may be similarly used, such as teeth, ridges, hooks, and the like.

Another aspect herein shown in FIG. 6 is retaining loop 660, that has been placed over and engages with the features of curved tips 615. Retaining loop 660 may be intended to simply retain force applying member 605 by preventing undesirable expansion, or alternatively, it may be used to further compress, cinch, or otherwise tighten force applying member around the anatomical structure during or after deployment. This optional feature provides the surgeon considerable flexibility to terms of being able place and ensure retention of device 600 around complex or potentially problematic anatomies, or to adjust the forces delivered by device 600 to the underlying tissue beyond that which can be achieved based on the geometry, dimensions and material properties of force applying member 605 alone. A variety of other features and mechanisms may be used in place of retaining loop 615 to achieve similar therapeutic results within the scope herein; for example, sutures, ties, springs, latches, interlocking features, ratchets, common mechanical connections, and the like may be used.

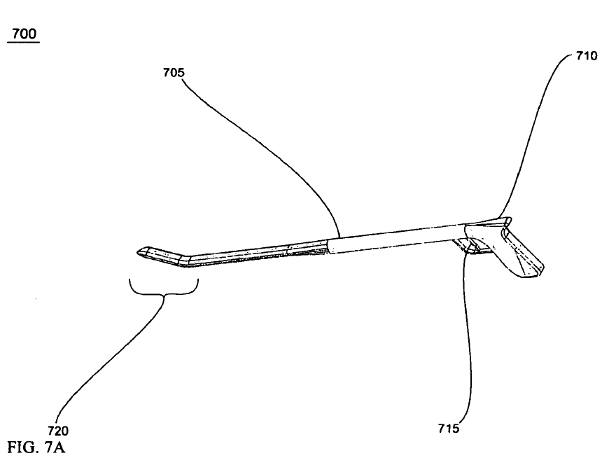

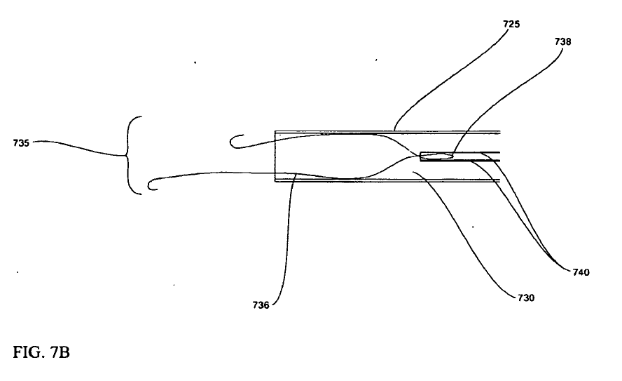

The devices discussed herein for treating the exterior of an anatomical structure are typically deployed, positioned, repositioned and removed using minimally invasive elements, wherein the applicators, deployment devices and/or innovative tools used for such functions in combination with the pressure applying-medical devices herein comprise exemplary systems herein. Such systems are typically configured and supplied to the surgeon as a kit containing all the components needed to successfully deliver the desired therapeutic result to the patient. An exemplary applicator device used for deploying the devices herein is shown in FIG. 7. In FIG. 7A, an exemplary minimally invasive applicator device 700, which can be a surgical device, comprises a longitudinal tubular assembly 705 within which one or more devices herein (e.g. device 100 of FIG. 1) are stored in the collapsed (i.e. pre-deployed) configuration. Positioned at the proximal end of longitudinal tube assembly 705 is handle assembly 710, wherein handle assembly 710 contains actuating mechanisms (not shown) that are operatively controlled using trigger 715. Positioned at the distal end of longitudinal tube assembly 705 and functionally connected to the actuating mechanisms within handle assembly 710 is deployment assembly 720. Deployment assembly 720 is capable of engaging and gripping a device herein as it is advanced distally along the axis of longitudinal tube assembly. Upon actuation of trigger 715 by the user, deployment assembly 720 is capable of further advancing the device herein longitudinally out of the distal end of applicator device 700, where it is then reconfigured to its expanded (i.e., deployed) configuration, as it is placed around the target anatomical structure.

FIG. 7B shows a close up schematic view of the cross section of the distal end of deployment assembly 720, according to one embodiment herein. Outer tube 725 contains working channel 730 within which the device to be deployed 735 is held and restrained in its collapsed (pre-deployed) configuration. In the example shown, device 735 is similar to device 300 (FIG. 3) wherein force applying member 736 incorporates asymmetric spring element 738 that assists device 735 to be gripped and advanced. Asymmetric spring element 738 is engaged by moveable arms 740 which are contained within working channel 730 and are operatively connected to the actuating mechanism in the handle assembly (not shown). During actuation by the user as illustrated in FIG. 7B, moveable arms 740 are advanced distally until device 735 begins to exit from working channel 730, at which point the force applying member 736 is gradually re-configured from its collapsed (pre-deployed) configuration into its expanded (deployed) configuration. At the same time, as moveable arms 740 are further advanced distally and extend out of working channel 730, they are used to guide and position device 735 at the desired placement location around the target anatomical structure (not shown). Upon release of asymmetric spring element 738 by moveable arms 740, force applying member 736 is fully re-configured and delivers the therapeutic compressive radial force to the exterior surface of the anatomical structure, according to the teachings herein.

### REFERENCES

1. Moore W S, Brewster D C, Bernhard V M. Aorto-uni-iliac endograft for complex aortoiliac aneurysms compared with tube/bifurcation endografts: results of the EVT/Guidant trials. J Vasc Surg 2001; 33:S11-20.

2. Zarins C K, White R A, Schwarten D, et al. AneuRx stent graft versus open surgical repair of abdominal aortic aneurysms: multicenter prospective clinical trial. J Vase Surg 1999; 29:292-305; discussion 306-8.

3. Carpenter J P, Anderson W N, Brewster D C, et al. Multicenter pivotal trial results of the Lifepath System for endovascular aortic aneurysm repair. J Vasc Surg 2004; 39:34-43.

4. Matsumura J S, Brewster D C, Makaroun M S, Naftel D C. A multicenter controlled clinical trial of open versus endovascular treatment of abdominal aortic aneurysm. J Vasc Surg 2003; 37:262-71.

5. Criado F J, Wilson E P, Fairman R M, Abul-Khoudoud O, Wellons E. Update on the Talent aortic stent-graft: a preliminary report from United States phase I and II trials. J Vasc Surg 2001; 33:S146-9.

6. Sternbergh W C, 3rd, Money S R, Greenberg R K, Chuter T A. Influence of endograft oversizing on device migration, endoleak, aneurysm shrinkage, and aortic neck dilation: results from the Zenith Multicenter Trial. J Vasc Surg 2004; 39:20-6.

7. Li Z, Kleinstreuer C. Effects of major endoleaks on a stented abdominal aortic aneurysm. J Biomech Eng 2006; 128:59-68.

8. Kudo, et.al. Laparoscopic aortic reinforcement and endovascular graft placement in swine: A new external wrap. Journal of Vascular Surgery. Volume 41, Number 6. 2006

From the foregoing, it will be appreciated that, although specific embodiments have been discussed herein for purposes of illustration, various modifications may be made without deviating from the spirit and scope of the discussion herein. Accordingly, the systems and methods, etc., include such modifications as well as all permutations and combinations of the subject matter set forth herein and are not limited except as by the appended claims or other claim having adequate support in the discussion herein.

1. A medical pressure device configured to apply a specifically controlled, compressive radial force to the exterior of an anatomical structure, the medical pressure device comprising at least one collapsible force-applying body member comprised of a surgically acceptable material, the force-applying body member having at least a first collapsed shape and a second expanded shape, wherein the first collapsed shape is sized for deployment by a minimally invasive applicator device, and the second expanded shape comprises a substantially U-shape configured to at least substantially encircle the anatomical structure and to provide a specifically controlled magnitude, orientation and spatial distribution of compressive radial force to the exterior of the anatomical structure.

2. The medical device of claim 1 wherein the force-applying body member comprises at least one collapsible element disposed between at least two substantially opposed arms.

3. The medical device of claim 1 wherein the force-applying body member is resiliently flexible.

4. The medical device of claim 1 wherein the force-applying body member comprises at least one collapsible element disposed between at least two substantially opposed, substantially rigid arms.

5. The medical device of claim 1 wherein the medical pressure device is curved to completely encircle the anatomical structure.

6. The medical device of claim 1 wherein the medical pressure device is curved to almost completely encircle the anatomical structure.

7. The medical device of claim 1 wherein the wherein the medical pressure device is sized and configured to substantially encircle the anatomical structure selected from the group consisting of an aorta.

8-11. (canceled)

12. The medical device of claim 1 wherein the device comprises at least one cooperative element configured to cooperatively interact with at least one other therapeutic device implanted in the anatomical structure.

13. The medical device of claim 12 wherein the at least one other therapeutic device comprises at least one of an endolumenal stent, an embolic coil, an artificial heart valve, and a gastrointestinal implant.

14. (canceled)

15. The medical device of claim 2 wherein the collapsible element is self-actuating such that it converts from the first shape to the second shape when the restraining pressure is removed without external input other than the removal of the restraining pressure.

16. The medical device of claim 2 wherein the collapsible element is a coil spring.

17. The medical device of claim 16 wherein the coil spring is asymmetrical.

18. The medical device of claim 1 wherein the force-applying body member comprises at least two substantially opposed arms, wherein at least one of the arms comprises at least one inwardly-facing anchoring element configured to engage the anatomical structure.

19-28. (canceled)

29. The medical device of claim 1 wherein the medical pressure device is at least one of a substantially flexible wire, rib, strap, band, sheet, wrap or cuff.

30. The medical device of claim 1 wherein the medical pressure device comprises at least one substantially opaque marker that is substantially opaque to at least one scanning visualization modality.

31. The medical device of claim 1 wherein the force-applying body member is composed of at least one of stainless steel, titanium, polymers, and superelastic NiTi alloy.

32. The medical device of claim 1 wherein the medical pressure device comprises at least one laterally-disposed interlocking element configured to interlockingly connect the medical pressure device to a second medical pressure device.

33. The medical device of claim 1 wherein the medical pressure device further comprises at least one connecting member configured to connect the medical pressure device to a second medical pressure device.

34. The medical device of claim 33 wherein the connecting member comprises at least one flexible biocompatible material comprising at least one of cloth, fabric, mesh, sheet and screen.

35. The medical device of claim 1 wherein the medical pressure device comprises at least one additional therapeutic element configured to provide a treatment to the anatomical structure in addition to the radial pressure.

36-75. (canceled)
